(19) United States
(12) Patent Application Publication
(10) Pub. No.: US 2011/0054014 A1
(43) Pub. Date: Mar. 3, 2011

(54) PRENYLFLAVANONE COMPOUNDS AND USES THEREOF

(75) Inventors: Chung-Yang Huang, Taipei City (TW); Chia-Nan Chen, Taipei City (TW); Wei-Jan Huang, Taipei City (TW); Li-Ling Chi, Taipei City (TW); Pen-Yuan Chen, Taipei City (TW); Chia-Wei Lin, Taipei City (TW)

(73) Assignee: NatureWise Biotech & Medicals Corporation, Taipei City (TW)

(21) Appl. No.: 12/942,719

(22) Filed: Nov. 9, 2010

Related U.S. Application Data
(62) Division of application No. 12/418,083, filed on Apr. 3, 2009.
Application No. 12/418,083 is a continuation-in-part of application No. 12/330,991, filed on Dec. 9, 2008.

(51) Int. Cl. A61K 31/353; A61P 25/28; A61P 25/00; A61P 35/00

(52) U.S. Cl. 514/456

(57) ABSTRACT

The present invention relates to new prenylflavanone compounds and a pharmaceutical composition comprising at least one of the compounds.

### Cross-Reference to Related Application

This application is a continuation-in-part and claims the benefit of U.S. application Ser. No. 12/330,991 filed on Dec. 9, 2008, the content of which is hereby incorporated by reference in its entity.

### BACKGROUND OF THE INVENTION

The preset invention relates to new prenylflavanone compounds and uses thereof.

Propolis is a resinous mixture that honeybees collect from tree buds, fruits, sap flows, or other botanical sources. It has been reported to possess various active components and exhibit a broad spectrum of biological activities, including antitumor⁽³⁾, antioxidant⁽⁴⁾, antibacterial⁽⁵⁾, antiviral⁽⁶⁾, antifungal⁽⁷⁾, and anti-inflammatory activities⁽⁸⁾. Propolis from different areas may contain different active components.

We previously reported eight prenylflavanones isolated from Taiwanese propolis which are represented by Formulae 1 to 8 as below, respectively.

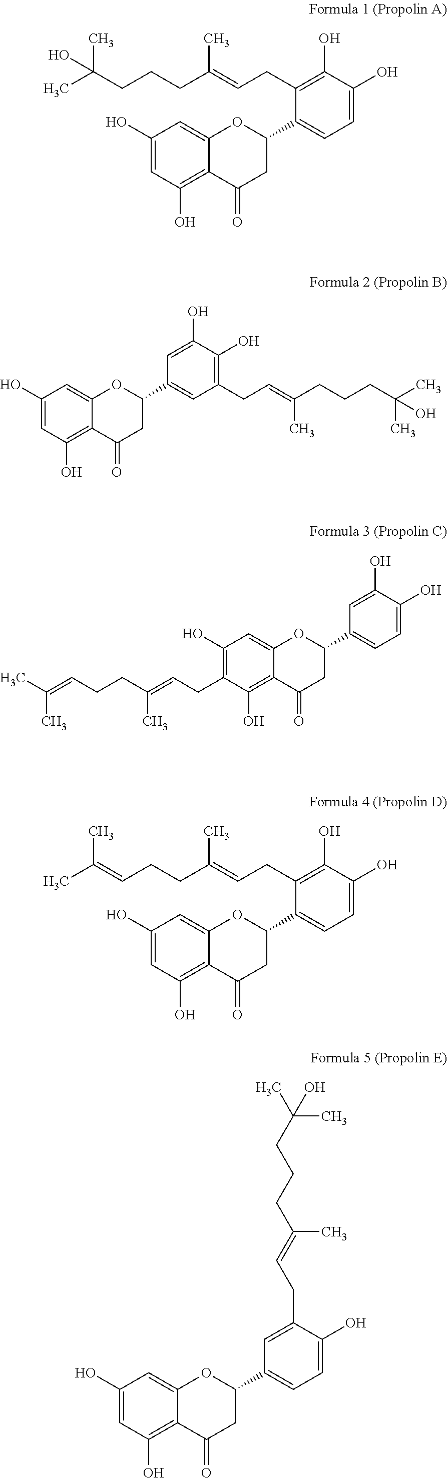

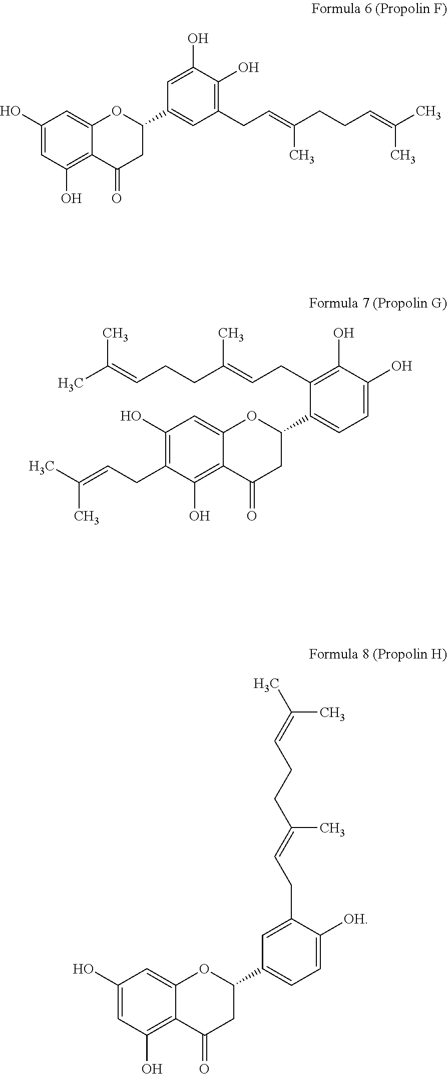

These propolins have been reported to exhibit a broad spectrum of biological activities, including anticancer⁽¹⁰⁻¹⁵⁾, antioxidant⁽¹⁰⁻¹⁶⁾ and antimicrobial activities⁽¹⁶⁾. In addition, recent studies demonstrated that Okinawan propolis contained active components similar to that of Taiwanese propolis⁽¹⁷⁻¹⁸⁾.

Histone deacetylase (HDAC) is an enzyme that catalyses deacetylation of the ε-amino group of lysine amino acid residues in the N-terminal tails of histones. Some HDAC inhibitors have been found and demonstrated to have therapeutic efficacy on patients suffered from different types of cancers in preclinical and clinical stages⁽¹⁹⁻²¹⁾. According to the current model for the anticancer mechanism of HDAC inhibitors, the inhibitors induce hyperacetylation of core histones and thus trigger chromatin remodeling and wake up silent genes such as tumor suppressor genes which result in inhibition of tumor cells growth⁽²⁶⁻²⁷⁾.

There is still a need to find new components from the multi-functional propolis and evaluate their useful physiological activities.

### BRIEF SUMMARY OF THE INVENTION

In one aspect, the present invention provides a new prenylflavanone compound which is represented by a formula selected from the group consisting of:

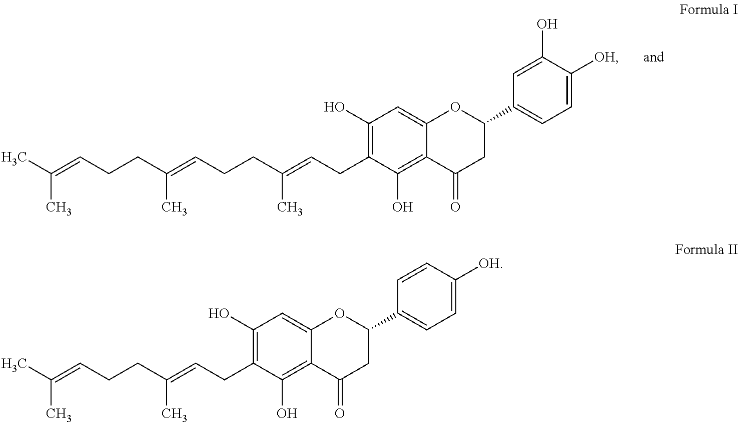

In another aspect, the present invention provides a pharmaceutical composition comprising at least one of the prenylflavanone compounds as describe above.

In yet another aspect, the present invention to provide a method for treating a disease or condition or providing a desired effect in a subject in need thereof comprising administrating to the subject at least one of the compounds as described above or the above-mentioned pharmaceutical composition. Particular embodiments are described below.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the preferred embodiments shown.

In the drawings:

FIG. 1 represents the key HMBC correlations between H and C in Propolin I.

FIG. 2 represents the key HMBC correlations between H and C in Propolin J.

FIG. 3 shows the peaks of the reversed-phase preparative HPLC in Example 2.

FIG. 4(A) shows the staining results of MCF-7 cells treated with different propolins at various concentrations for 24 hours, wherein (a) refers to the control treatment; (b), (c) and (d) refer to the treatment by Propolin F at a concentration of 5, 10 and 15 μg/mL, respectively; (e), (f) and (g) refers to the treatment by Propolin I at a concentration of 2.5, 5.0 and 7.5 μg/mL, respectively; and (h), (i) and (j) refer to the treatment by Propolin J at a concentration of 5, 10 and 15 μg/mL.

FIG. 4(B) shows the staining results of MDA-MB-231 cells treated with different propolins at various concentrations for 24 hours, wherein (a) refers to the control treatment; (b), (c) and (d) refer to the treatment by Propolin F at a concentration of 5, 10 and 15 μg/mL, respectively; (e), (f) and (g) refers to the treatment by Propolin I at a concentration of 2.5, 5.0 and 7.5 μg/mL, respectively; and (h), (i) and (j) refer to the treatment by Propolin J at a concentration of 5, 10 and 15 μg/mL, respectively.

FIG. 4(C) shows the staining results of MCF-7 cells treated with different propolins at various concentrations for 72 hours, wherein (a) refers to the control treatment; (b), (c) and (d) refer to the treatment by Propolin F at a concentration of 5, 10 and 15 μg/mL, respectively; (e), (f) and (g) refer to the treatment by Propolin I at a concentration of 2.5, 5.0 and 7.5 μg/mL, respectively; and (h), (i) and (j) refer to the treatment by Propolin J at a concentration of 5, 10 and 15 μg/mL, respectively.

FIG. 4(D) shows the staining results of MDA-MB-231 cells treated with different propolins at various concentrations for 72 hours, wherein (a) refers to the control treatment; (b) and (c) refer to the treatment by Propolin F at a concentration of 10 and 15 μg/mL, respectively; and (d) and (e) refer to the treatment by Propolin I at a concentration of 5.0 and 7.5 μg/mL, respectively; and (f) and (g) refer to the treatment by Propolin J at a concentration of 10 and 15 μg/mL, respectively.

FIG. 4(E) shows the numbers of MCF-7 cells treated with different propolins at various concentrations for 72 hours.

FIG. 5(A) shows the result of the flow cytometric assay for the MCF-7 cells treated with different propolins at various concentrations for 72 hours, wherein (a) refers to the control treatment; (b), (c) and (d) refer to the treatment by Propolin F at a concentration of 5, 10 and 15 μg/mL, respectively; (e), (f) and (g) refers to the treatment by Propolin I at a concentration of 2.5, 5.0 and 7.5 μg/mL, respectively; and (h), (i) and (j) refer to the treatment by Propolin J at a concentration of 5, 10 and 15 μg/mL.

FIG. 5(B) shows the result of the flow cytometric assay for the MDA-MB-231 cells treated with different propolins at various concentrations for 24 hours, wherein (a) refers to the control treatment; (b), (c) and (d) refer to the treatment by Propolin F at a concentration of 5, 10 and 15 μg/mL, respectively; (e), (f) and (g) refers to the treatment by Propolin I at a concentration of 2.5, 5.0 and 7.5 μg/mL, respectively; and (h), (i) and (j) refer to the treatment by Propolin J at a concentration of 5, 10 and 15 μg/mL.

FIG. 5(C) shows the result of the flow cytometric assay for the MDA-MB-231 cells treated with different propolins at various concentrations for 72 hours, wherein (a) refers to the control treatment; (b) and (c) refer to the treatment by Propolin F at a concentration of 10 and 15 μg/mL, respectively; and (d) and (e) refer to the treatment by Propolin I at a concentration of 5.0 and 7.5 μg/mL, respectively; and (f) and (g) refer to the treatment by Propolin J at a concentration of 10 and 15 μg/mL, respectively.

FIG. 6(A) shows the results of the immunocytochemistry study in Example 6.

FIG. 6(B) shows the results of the western blotting assay in Example 6.

### DETAILED DESCRIPTION OF THE INVENTION

Unless defined otherwise, all technical and scientific terms used herein have the same meanings as is commonly understood by one of skill in the art to which this invention belongs.

In one aspect, the present invention provides a prenylflavanone compound isolated from Taiwanese propolis.

One embodiment of the prenylflavanone compound is 5,7,3′,4′-tetrahydroxy-6-farnesylflavanone (named “Propolin I”), which is represented by the following formula:

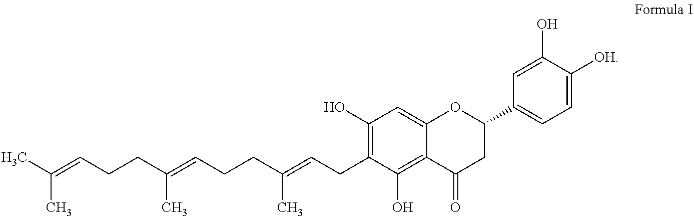

The compound of Formula I has the molecular formula of C₃₀H₃₆O₆, and a molecular weight of 492.25 Da.

Another embodiment of the prenylflavanone compound is 5,7,4′-trihydroxy-6-geranylflavanone (named “Propolin J”), which is represented by the following formula:

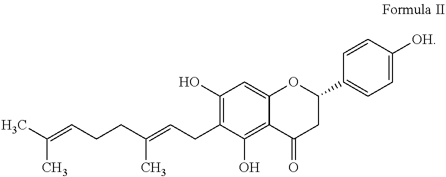

The compound of Formula II has the formula of C₂₅H₂₈O₅ and a molecular weight of 408.19 Da.

The above-mentioned prenylflavanone compounds of the invention have inhibitory effects on HDAC enzymatic activity and growth of cancer cells, preferably that of breast cancer cells.

Accordingly, in another aspect, the present invention provides a pharmaceutical composition as a HDAC inhibitor comprising at least one of the compounds of the invention and a pharmaceutical acceptable excipient or carrier.

In yet another aspect, the present invention provides a pharmaceutical composition for inhibiting cancer cell growth comprising at least one of the compounds of the invention and a pharmaceutically acceptable excipient or carrier.

The present invention also provides a pharmaceutical composition for treating a disease in association with histone deacetylation comprising at least one of the compounds of the invention and a pharmaceutically acceptable excipient or carrier.

In addition, many of known HDAC inhibitors have been demonstrated to have neuroprotective effects^{(30, 31)}, implying that a HDAC inhibitor is useful in the treatment of neurodegenerative diseases. Examples of the neurodegenerative diseases include, but are not limited to, multiple sclerosis (MS)⁽³²⁾, Huntington's disease (HD)^{(33, 39, 40)}, spinal muscular atrophy (SMA)^{(34, 35, 36)}, spinal and bullar muscular atrophy (SBMA)⁽³⁷⁾, and amyotrophic lateral sclerosis (ALS)⁽³⁸⁾.

Accordingly, the present invention thus relates to a pharmaceutical composition having neuroprotective effects comprising at least one of the compounds of the invention and a pharmaceutically acceptable excipient or carrier.

The present invention also relates to a pharmaceutical composition for treating a neurodegenerative disease comprising at least one of the compounds of the invention and a pharmaceutically acceptable excipient or carrier. Preferably, the neurodegenerative disease is multiple sclerosis (MS), Huntington's disease (HD), spinal muscular atrophy (SMA), spinal and bullar muscular atrophy (SBMA), or amyotrophic lateral sclerosis (ALS).

In making the composition of the invention, the compound of the invention as the active ingredient is usually diluted with an excipient or enclosed within a carrier which can be in the form of a capsule, sachet, paper or other container. The excipient employed is typically an excipient suitable for administration to human subjects or other mammals. Some examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. In addition, the composition of the invention can be in the form of tablets, pills, powders, suspensions, emulsions, solutions, syrups, aerosols (as a solid or in a liquid medium), ointments, capsules, or sterile packaged powders.

The compositions can be administered to a subject including humans, monkeys, dogs, etc. by an oral route, for example, or they can be administered by a parenteral route in the form of injectable preparations. Typically the composition of the invention contains from about 125 to about 250 mg of the compound of the invention (Propolins I or J, or both). The dose for adults is preferably between about 500 and about 1000 mg per day, which can be administered in a single dose or in the form of separated doses.

In another aspect, the present invention provides a method for treating a disease or condition or providing a desired effect in a subject in need thereof comprising administrating to the subject at least one of the compounds as described above or the above-mentioned pharmaceutical composition.

As used herein, a subject in need of the treatment according to the invention includes human and non-human mammals. Non-human mammals include, but are not limited to, companion animals such as cats, dogs and the like and farm animals such as cattle, horses, sheep, goats, swine and the like.

In particular, the above-mentioned method of the invention is useful in inhibiting cancer cell growth in a subject. More particular, the method of the invention is useful in treating growth of breast cancer cells in a subject.

In addition, the method of the invention is useful in treating a disease in association with histone deacetylation. Moreover, the method of the invention can provide neuroprotective effects in a subject.

Furthermore, the method of the invention is useful in treating a neurodegenerative disease in a subject. Specifically, the neurodegenerative disease is selected from the group consisting of multiple sclerosis (MS), Huntington's disease (HD), spinal muscular atrophy (SMA), spinal and bullar muscular atrophy (SBMA), and amyotrophic lateral sclerosis (ALS).

The present invention will now be described more specifically with reference to the following embodiments, which are provided for the purpose of demonstration rather than limitation.

### Example 1

### Primary Extraction

200 g of Taiwanese propolis of TW-I grade (collected from hives located in Tainan, Taiwan)⁽²⁹⁾ was homogenized by stirring at a low temperature. The homogenized sample was then washed 3 times with 1.0 L of deionized water and the residue was extracted 3 times with 95% ethanol. The filtered ethanol extract was evaporated to dryness under reduced pressure and a brown powder was obtained (135.6 g) which was stored at −20° C. until further purification.

### Example 2

### Isolation and Purification of Compounds

The brown powder obtained from the ethanol extract was dissolved in methanol and applied to a Sephadex LH-20 column (Amersham Pharmacia Biotech AB, Uppsala, Sweden) using 95% ethanol as the eluting solvent. All the elutes, including the fractions obtained by the follow-up chromatographies, were assayed for their effects on human breast cancer proliferation, and the active fractions were again separated by chromatographies on a Sephadex LH-20 column using 95% ethanol as the eluting agent. Next, the active fractions were subjected to silica gel column chromatography (Kiesel gel 60, E. Merck, Darmstadt 1, Germany) using an n-hexane/EtOAc solvent system.

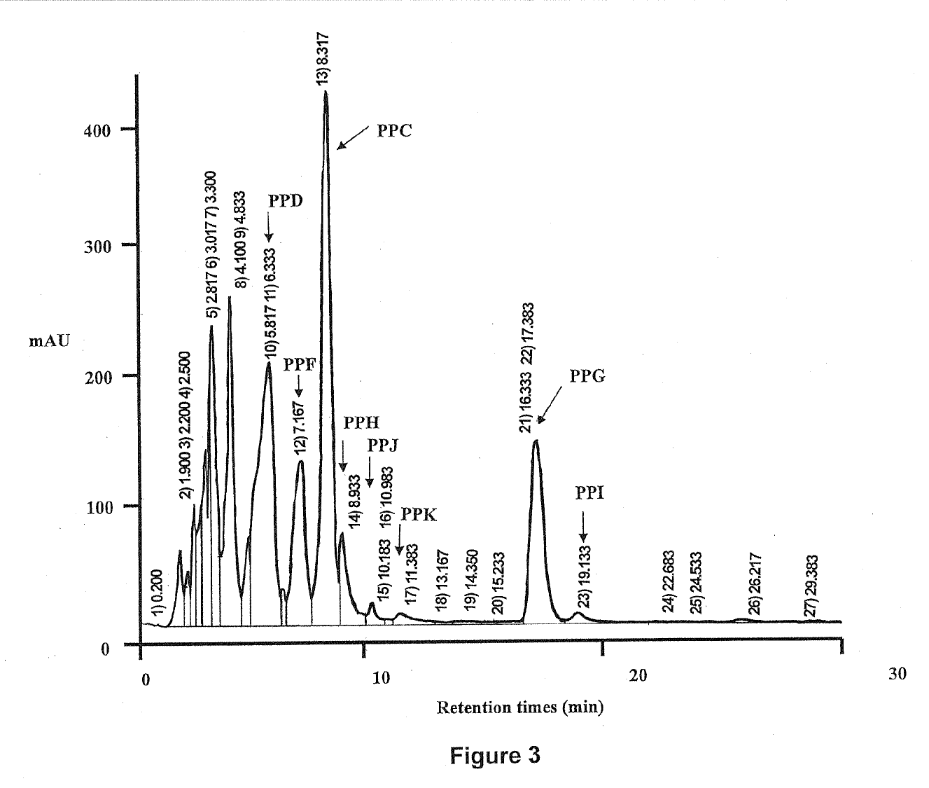

The purification of the most active fraction was performed by reversed-phase preparative HPLC (n-hexane/EtOAc, 30:70). The experimental conditions were as follows: the column was Luna Phenomenex (C18, 250×4.6 mm); the solvent system was methanol/water (8:2); the flow rate was 1 mL/min; and the detection was conducted at UV 280 nm. Fractions at the retention time, 19.0 min (Propolin I) and 10.5 min (Propolin J), were collected whereby Propolin I (light yellow powder) and Propolin J (light yellow liquid) were isolated, respectively. FIG. 3 shows the peaks of the reversed-phase preparative HPLC.

### Example 3

### Identification of Propolin I and Propolin J

The structure of each of Propolins I and J was analyzed by using the following instruments: Perkin Elmer 1760-X IR-FT spectrometer; Hitachi 150-20 UV; Jasco J-710 spectropolarimeter; Finnigan MAT-95XL mass spectrometer (EI); and Bruker AV-500 spectrometer using the solvent peak (MeOH-d3) as a reference standard.

### Propolin I

The physicochemical data of Propolin I is shown as follows:

[ ]²⁰_{D}+3.8 (c 0.26, CH₃OH); IR νmax (film) 3747, 3390, 2923, 1636 cm⁻¹; UV (EtOH) λmax nm (log ε) 207.0 (1.65), 291 (0.73); CD (MeOH) 347 nm (Δε +3.19), 294 nm (Δε −3.98); HREIMS m/z 492.2512 (calcd for C₃₀H₃₆O₆ 492.2512); ¹H and ¹³C NMR.

From the ¹H NMR spectrum, it was confirmed that there were four olefinic methyl groups (δ_{H} 1.52, 1.56, 1.63, 1.74), eight methylene protons (δ_{H} 1.87, 1.95, 2.05), two benzylic methylene protons (δ_{H} 3.20) and two vinyl protons (δ_{H} 5.04, 5.18), which indicated the presence of a farnesyl group. Besides, the ¹³C NMR spectrum showed that there were four methyl groups (δ_{c} 16.1, 16.2, 17.8, 25.9), five methylene carbons (δ_{c} 21.8, 27.3, 27.8, 40.8, 40.9), three tertiary olefinic carbons (δ_{c} 124.1, 125.3, 125.6) and three quaternary olefinic carbons (δ_{c} 132.0, 134.9, 135.8) which further supported the presence of a farnesyl group. Moreover, the ABX system at δ_{H} 5.21 (1H, dd, J=3.0, 13.0 Hz), 2.65 (1H, dd, J=3.0, 17.0 Hz), and 3.02 (1H, dd, J=13.0, 17.0 Hz) displayed a characteristic pattern for a flavanone skeleton at the H-2 and H-3 position, respectively.

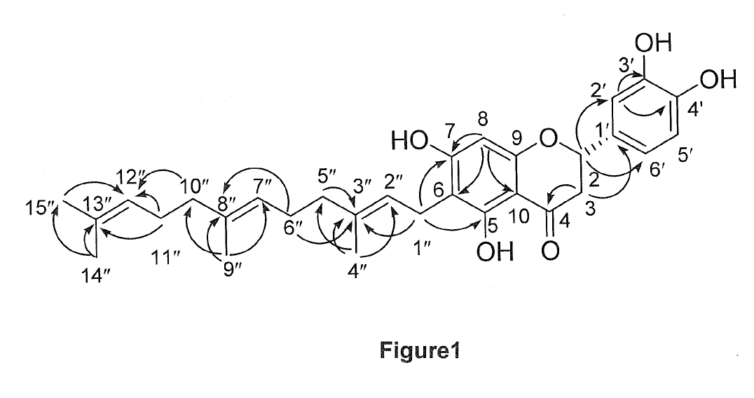

In addition, the total ¹H and ¹³C NMR assignments and connectivities were determined based on the inference of the ¹H—¹H COSY, HSQC, and HMBC data. The HMBC spectrum of Propolin I revealed that the methylene signal at δ_{H} 3.20 (H-1″) was correlated with C-5 (δ_{c} 162.5) and C-7 (δ_{c} 165.9), respectively, which suggested that the farnesyl group was attached to C-6 (FIG. 1). In addition, its CD spectra indicated that the configuration of C-2 was S-form.

Accordingly, Propolin I was identified as 5,7,3′,4′-tetrahydroxy-6-farnesylflavanone, which is represented by Formula I as mentioned above. This compound was isolated for the first time and had not been disclosed in published literatures.

### Propolin J

The physicochemical data of Propolin J is shown as follows:

[ ]²⁰_{D}+2.4 (c 0.41, CH₃OH); IR νmax (film) 3747, 3393, 2925, 2361, 1637 cm⁻¹; UV (EtOH) λmax nm (log ε) 209.0 (1.82), 293 (0.92); CD (MeOH) 328.9 nm (Δε +2.37), 289 nm (Δε −3.98); HREIMS m/z 408.1944 (calcd for C₂₅H₂₈O₅ 408.1937); ¹H and ¹³C NMR.

From the ¹H NMR spectrum, it was confirmed that there were three olefinic methyl groups (δ_{H} 1.55, 1.61, 1.74), four methylene protons (δ_{H} 1.93, 2.04), two benzylic methylene protons (δ_{H} 3.18) and two vinyl protons (δ_{H} 5.05, 5.18), which indicated the presence of a geranyl group. Besides, the ¹³C NMR spectrum shown that there were three methyl groups (δ_{c} 16.2, 17.7, 25.9), three methylene carbons (δ_{c} 21.8, 27.7, 40.9), two tertiary olefinic carbons (δ_{c} 123.9, 125.5), and two quaternary olefinic carbons (δ_{c} 132.0, 135.3), which further supported the presence of a geranyl group. Moreover, the ABX system at δ_{H} 5.29 (1H, dd, J=2.7, 13.0 Hz), 2.67 (1H, dd, J=2.7, 17.0 Hz), and 3.08 (1H, dd, J=13.0, 17.0 Hz) displayed a characteristic pattern for a flavanone skeleton at H-2 and H-3 positions, respectively.

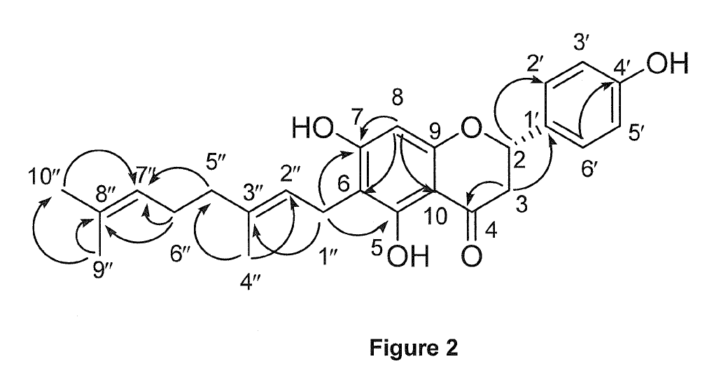

In addition, the total ¹H and ¹³C NMR assignments and connectivities were determined based on the inference of the ¹H—¹H COSY, HSQC, and HMBC data. The HMBC spectrum of Propolin J revealed that the methylene signal at δ_{H} 3.18 (H-1″) correlated with C-5 (δ_{c} 162.5) and C-7 (δ_{c} 166.0), which suggested that the geranyl group was attached to C-6 (FIG. 2). In addition, its CD spectra indicated that the configuration of C-2 was S-form.

Accordingly, Propolin J was identified as 5,7,4′-trihydroxy-6-geranylflavanone, which is represented by Formula II as mentioned above. This compound was isolated for the first time and had not been disclosed in published literatures.

Table 1 lists the physicochemical data of Propolins I and J.

TABLE 1

| | Propolin I | | Propolin J | |
|---|---|---|---|---|
| position | δ_{C}, multi. | δ_{H} (J in Hz) | δ_{C}, multi. | δ_{H} (J in Hz) |
| 2 | 80.5, CH | 5.21, dd (3.0, 13.0) | 80.4, CH | 5.29, dd (2.7, 13.0) |
| 3 | 44.3, CH₂ | 2.65, dd (3.0, 17.0) | 44.2, CH₂ | 2.67, dd (2.7, 17.0) |
| | | 3.02, dd (13, 17.0) | | 3.08, dd (13, 17.0) |
| 4 | 197.8, qC | | 197.9, qC | |
| 5 | 162.5, qC | | 162.5, qC | |
| 6 | 109.7, qC | | 109.7, qC | |
| 7 | 165.9, qC | | 166.0, qC | |
| 8 | 95.4, CH | 5.92, s | 95.4, CH | 5.93, s |
| 9 | 162.4, qC | | 162.4, qC | |
| 10 | 103.2, qC | | 103.2, qC | |
| 1′ | 131.9, qC | | 131.2, qC | |
| 2′ | 114.7^{a}, CH | 6.90, s | 129.0, CH | 7.30, d (8.6) |
| 3′ | 146.5^{b}, qC | | 116.3, CH | 6.80, d (8.6) |
| 4′ | 146.8^{b}, qC | | 159.0, qC | |
| 5′ | 116.2, CH | 6.77, s | 116.3, CH | 6.80, d (8.6) |
| 6′ | 119.2^{a}, CH | 6.77, s | 129.0, CH | 7.30, d (8.6) |
| 1″ | 21.8, CH₂ | 3.20, d (7.2) | 21.8, CH₂ | 3.18, d (7.1) |
| 2″ | 124.1, CH | 5.18, t (5.1) | 123.9, CH | 5.18, t (7.1) |
| 3″ | 134.9, qC | | 135.3, qC | |
| 4″ | 16.1, CH₃ | 1.74, s | 16.2, CH₃ | 1.74, s |
| 5″ | 40.8, CH₂ | 1.95, m | 40.9, CH₂ | 1.93, m |
| 6″ | 27.3, CH₂ | 2.05, dd (6.9, 14.0) | 27.7, CH₂ | 2.04, dd (6.9, 14.9) |
| 7″ | 125.3, CH | 5.04, m | 125.5, CH | 5.05, t (7.2) |
| 8″ | 135.8, qC | | 132.0, qC | |
| 9″ | 16.2, CH₃ | 1.52, s | 25.9, CH₃ | 1.61, s |
| 10″ | 40.9, CH₂ | 1.87, m | 17.7, CH₃ | 1.55, s |
| 11″ | 27.8, CH₂ | 1.95, m | | |
| 12″ | 125.6, CH | 5.04, m | | |
| 13″ | 132.0, qC | | | |
| 14″ | 25.9, CH₃ | 1.63, s | | |
| 15″ | 17.8, CH₃ | 1.56, s | | |

^{a,b}Data interchangeable.

### Example 4

### Cell Culture and Cytotoxicity Assay

Human breast cancer MCF-7 and MDA-MB-231 cells were purchased from the Food Industry Research and Development Institute (Hsinchu, Taiwan). The cells were cultured in Dulbecco's modified Eagle's medium (Gibco) containing 10% fetal bovine serum (FBS), a 1% dilution of penicillin-streptomycin, and 2 mM glutamine. Cells were maintained at 37° C. in a humidified atmosphere of 95% air and 5% CO₂. However, MDA-MB-231 cells were cultured in L-15 medium (Gibco) containing 10% fetal bovine serum (FBS), a 1% dilution of penicillin-streptomycin, and 2 mM glutamine. Cells were maintained at 37° C. in a humidified atmosphere of 100% air and 0% CO₂.

The propolins F, I, and J were dissolved in dimethyl sulfoxide (DMSO) and prepared at a fixed concentration of 10 mg/mL. The cells (1.5×10⁶ per dish) were cultured in a 100-mm dish and incubated for 14 h prior to treatment with DMSO or with different concentrations of propolins (2.5, 5.0, 10, and 20.0 μg/mL) for 48 h. The propolins were small compounds with their MWs ranging from 408-492 Da. These values were converted to their respective molarities.

The cells were counted and their viability was determined by trypan blue exclusion assay. The activity was shown as IC₅₀ value (the concentration required to induce 50% cytotoxicity), and the average value was obtained from triplicate data points.

Table 2 shows the IC₅₀ (μM) values of Propolins I and J of the invention and that of Propolin F as a control obtained in the cytotoxic assay.

TABLE 2

| Compounds | IC₅₀ values^{a} |
|---|---|
| Propolin F | 47.2 |
| Propolin I | 10.2 |
| Propolin J | 36.8 |

^{a}IC50 (μM) values were from one representative experiment of three independent experiments.

Accordingly, the compounds of the invention (Propolins I and J) have been found to have cytotoxic activity against human breast cancer cells.

### Example 5

### Inhibitory Effect of Propolins I and J on Growth of Cancer cells

### Cell Staining

Two types of breast cancer cell lines, MCF-7 (ER receptor positive) and MDA-MB-231 cells (ER receptor negative), were cultured in the conditions as described in Example 5. The cells were treated with different propolins at various concentrations for 24 or 72 hours. The treated cells were stained with trypan blue and evaluated for viability.

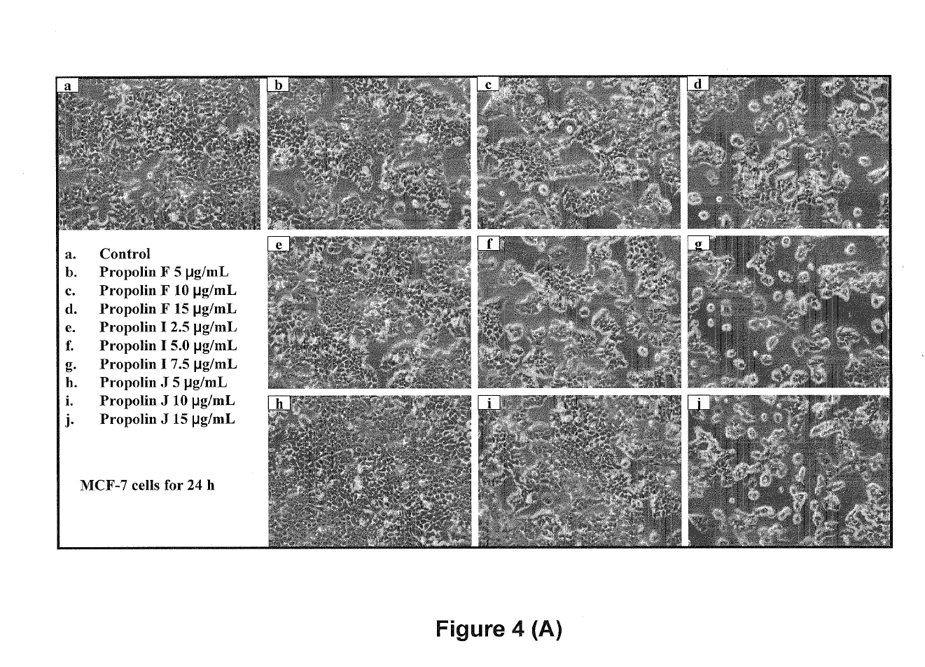

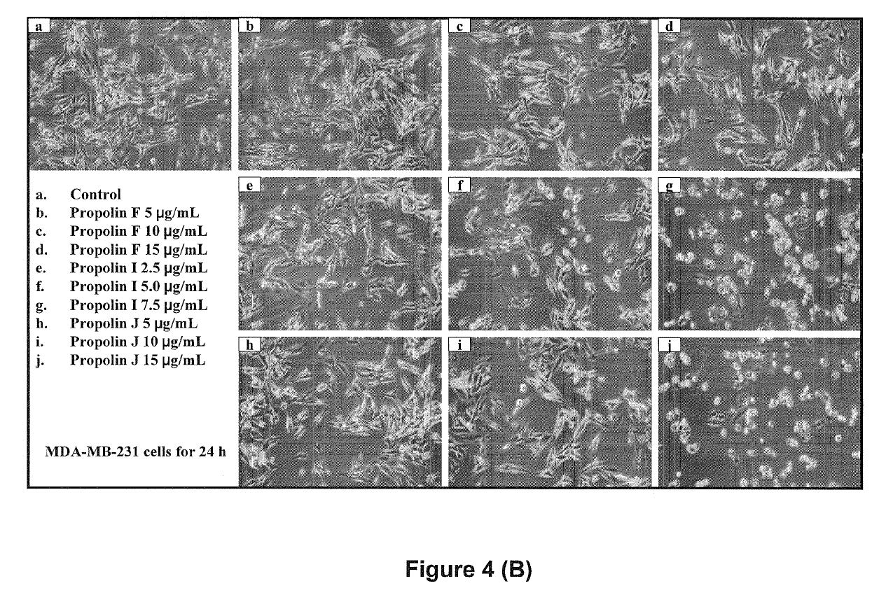

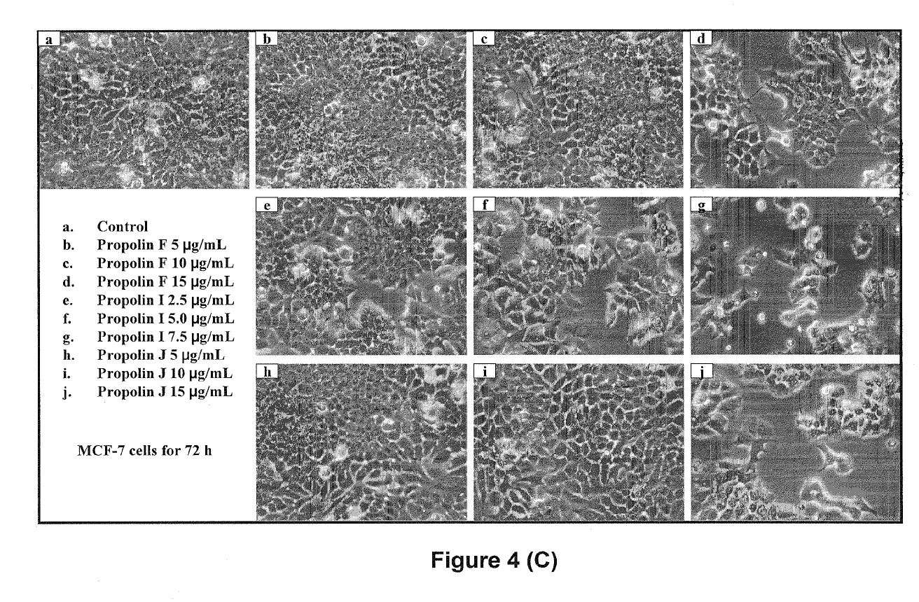

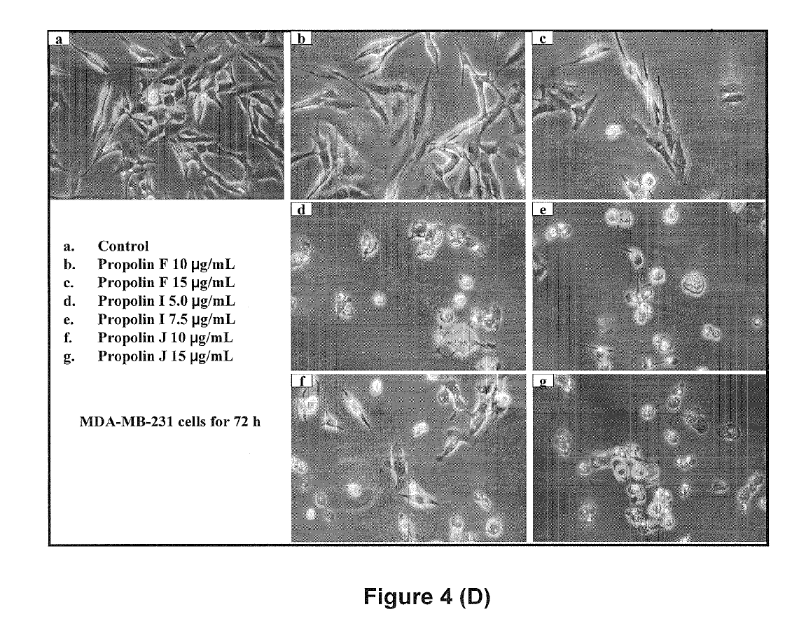

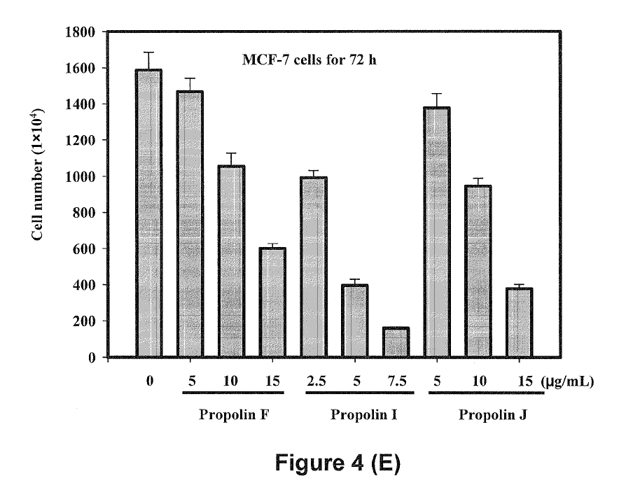

FIG. 4(A) shows the staining results of MCF-7 cells treated with the propolins at various concentrations for 24 hours. FIG. 4(B) shows the staining results of MDA-MB-231 cells treated with the propolins at various concentrations for 24 hours. FIG. 4(C) shows the staining results of MCF-7 cells treated with the propolins at various concentrations for 72 hours. FIG. 4(D) shows the staining results of MDA-MB-231 cells treated with the propolins at various concentrations for 72 hours. FIG. 4(E) shows the cell numbers of MCF-7 cells treated with the propolins at various concentrations for 72 hours.

### Flow Cytometric Analysis

Human breast cancer MCF-7 and MDA-MB-231 cells (1.5×10⁶) in a 100-mm dish were treated with various concentrations of propolins F, I, and J for 24 or 72 h. Cells were trysinized and collected with ice cold PBS. The cells were resuspended in 200 μL PBS and fixed by adding 800 μL of iced 100% ethanol then incubated overnight at −20° C. The cell pellets were collected by centrifugation, resuspended in 1 mL of hypotonic buffer (0.5% Triton X-100 in PBS and 1 μg/mL RNase A), and incubated at 37° C. for 30 min. Then, 1 mL of PI solution (50 μg/mL) was added, and the mixture was allowed to stand at 4° C. for 30 min. Cellular DNA content was then analysed by FACScan cytometry (Becton Dickinson).

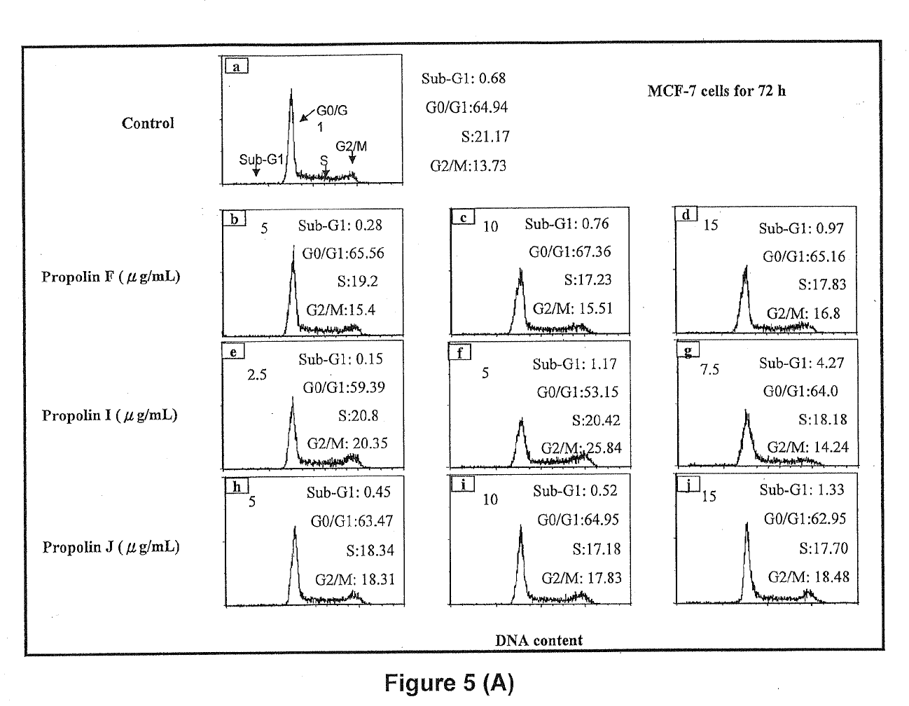

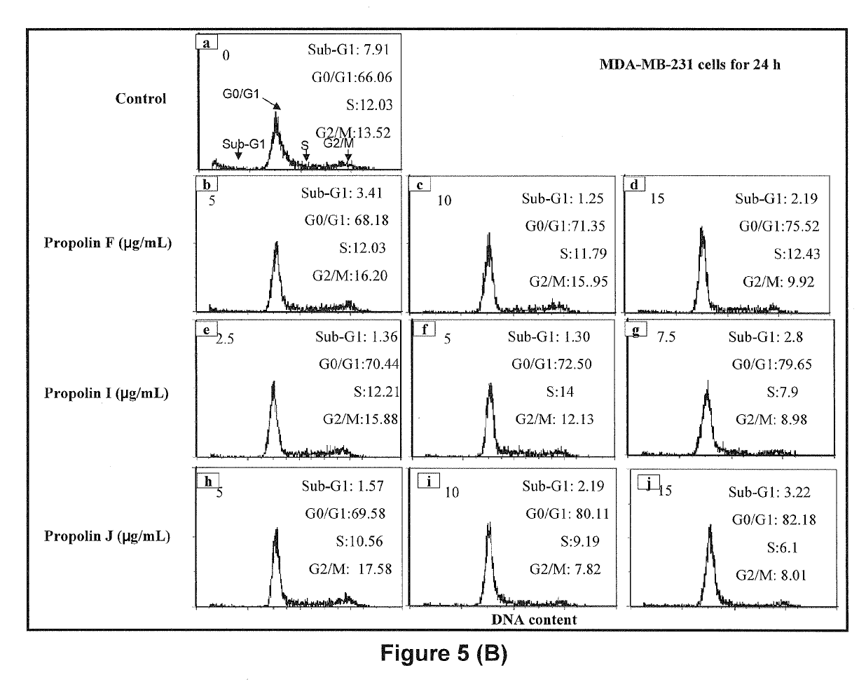

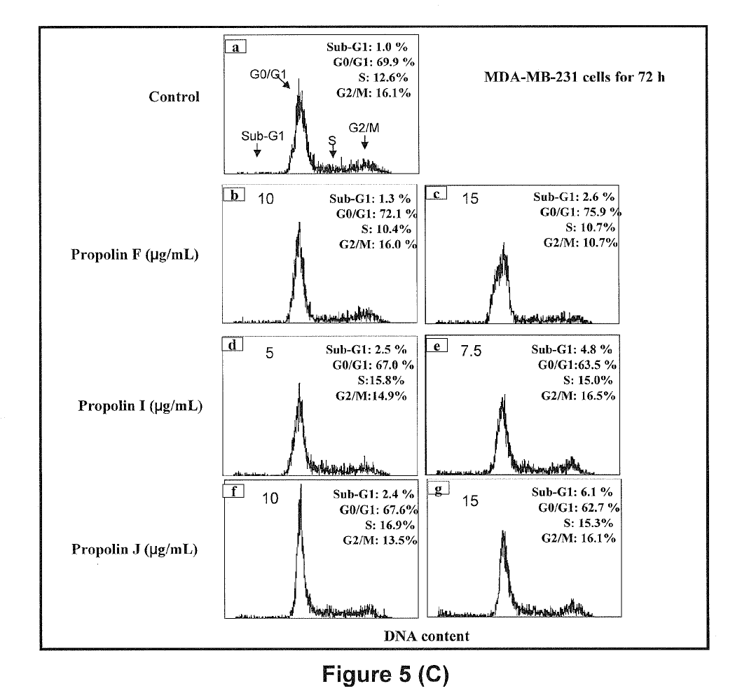

FIG. 5(A) shows the result of MCF-7 cells treated with the propolins at various concentrations for 72 hours. FIG. 5(B) shows the result of MDA-MB-231 cells treated with the propolins at various concentrations for 24 hours. FIG. 5(C) shows the result of MDA-MB-231 cells treated with the propolins at various concentrations for 24 hours.

As shown in FIGS. 4 and 5, the compounds of the invention were demonstrated having inhibitory effect on growth of the cancer cells.

### Western Blotting Assay

Human breast cancer MCF-7 cells (1.5×10⁶) on 100-mm dishes were treated with propolins F, I, and J at various concentrations for 24 h. After treatment, cells were collected and resuspended in 100 μl lysis buffer. Equal amounts of proteins (30 μg), were mixed with 2× sample buffer and resolved by 12.5% SDS-PAGE PAGE for β-actin, Bid, p21, Ac-histone 3, CTPS, and gelsolin detection. Proteins were electrotransferred to an immobilon membrane (PVDF; Millipore Corp.), and equivalent protein loading was verified by staining the membrane with reversible dye amido black (Sigma Chemical Co.). This was followed by overnight blocking with a solution composed of 20 mM Tris-HCl (pH 7.4), 125 mM NaCl, 0.2% Tween 20, and 3% BSA. Specific antibodies used were anti-human Bid (1:500 of rabbit polyclonal; Cell Signaling Technology, Inc.), anti-Ac-histone 3 (1:1000 of rabbit polyclonal; Cell Signaling Technology, Inc.), anti-p21 (1:1000 of mouse monoclonal; BD Pharmingen Technology, Inc.), anti-CTPS (1:1000 of mouse monoclonal; ABNOVA TAIWAN Corporation), gelsolin (1:1000 of mouse monoclonal; Sigma Chemical Co.), and anti-β-actin (1:5000 of mouse monoclonal; Cell Signaling Technology, Inc.). These proteins were detected by chemiluminescence (ECL, Amersham).

### Example 6

### HDAC Enzymatic Activity Assay

### Immunocytochemistry Study

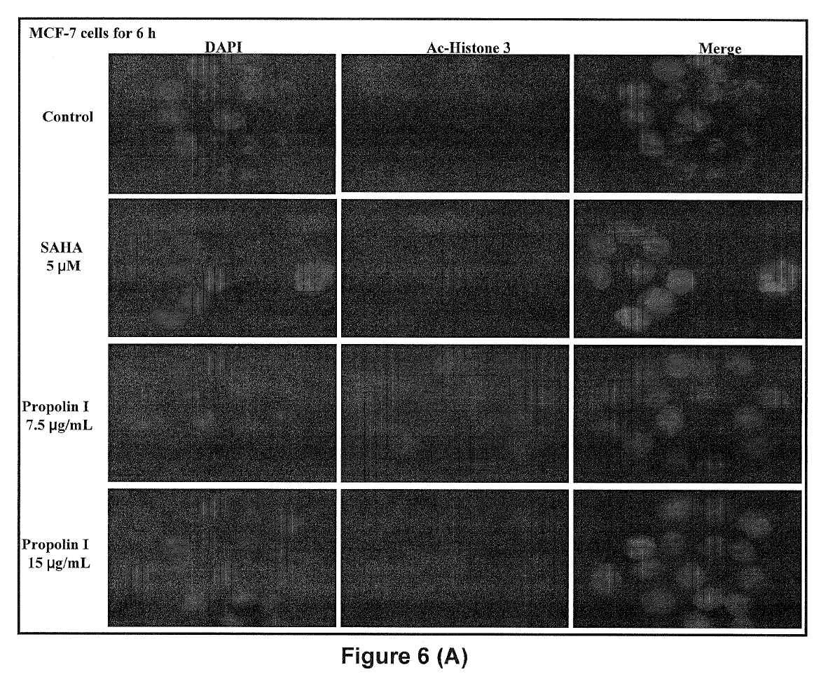

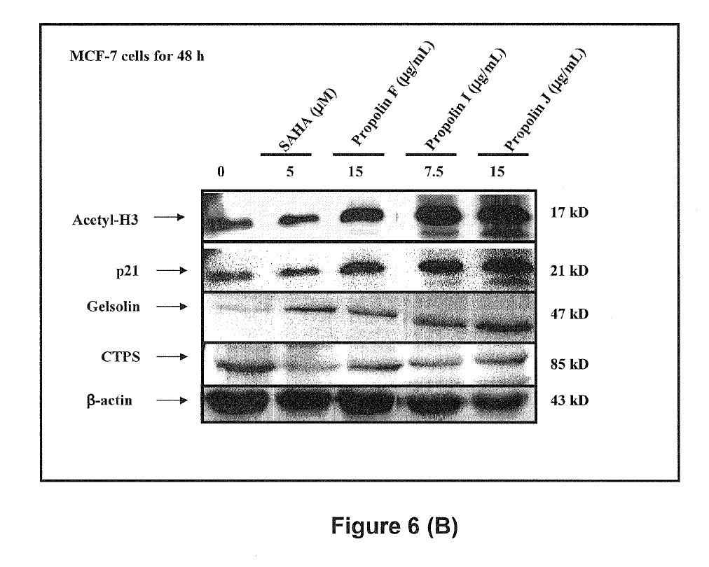

MCF-7 cells were cultured on six-well of culture slides and treated with propolins F, I, and J for 6 h. SAHA known as a HDAC inhibitor was used as a positive control. Slides were fixed for 30 minute at room temperature in a solution of 80% methanol and then washed 3 times in PBS. Cells were permeabilized for 30 minutes at room temperature with 0.3% Triton X-100, and then blocked in 10% fetal bovine serum (FBS) in PBS for 1 hour at room temperature before incubating overnight at 4° C. with antiacetylated histone H3 (Cell Signaling) diluted 1:500. After washing 3 times in PBS, the slides were stained with antirabbit IgG—conjugated secondary antibody for 1.0 hours then washed 3 times in PBS, and mounted with mounting medium (Sigma). In a parallel control experiment, it was observed that omission of either primary antibody eliminated staining Slides were analyzed by means under a fluorescence microscope with an Olympus PM30 camera (Melville, N.Y.). FIG. 6(A) shows the results of the immunocytochemistry study.

### Western Blotting Assay

The western blotting assay was carried out for Ac-histone 3, p21, gelsolin, CTPS and β-actin according to the method as described in Example 6. FIG. 6(B) shows the result of the western blotting assay.

As shown in FIGS. 6(A) and (B), the compounds of the invention were proved having inhibitory effects on HDAC enzymatic activity.

### REFERENCES

(1) Burdock, G. A. Review of the biological properties and toxicity of bee propolis (propolis). Food Chem Toxicol. 1998, 36, 347-363.

(2) Teixeira, E. W.; Negri, G.; Meira, R. M.; Message, D.; Salatino, A. Plant Origin of Green Propolis: Bee Behavior, Plant Anatomy and Chemistry. Evid Based Complement Alternat Med. 2005, 2, 85-92.

(3) Ahn, M. R.; Kunimasa, K.; Ohta, T.; Kumazawa, S.; Kamihira, M.; Kaji, K.; Uto, Y.; Hori, H.; Nagasawa, H.; Nakayama, T. Suppression of tumor-induced angiogenesis by Brazilian propolis: major component artepillin C inhibits in vitro tube formation and endothelial cell proliferation. Cancer Lett. 2007, 252, 235-243.

(4) Altu\check{g}, M. E.; Serarslan, Y.; Bal, R.; Konta [symbol] , T.; Ekici, F.; Melek, I. M.; Aslan, H.; Duman, T. Caffeic acid phenethyl ester protects rabbit brains against permanent focal ischemia by antioxidant action: a biochemical and planimetric study. Brain Res. 2008, 1201, 135-142.

(5) Drago, L.; De Vecchi, E.; Nicola, L.; Gismondo, M. R. In vitro antimicrobial activity of a novel propolis formulation (Actichelated propolis). J Appl Microbiol. 2007, 103, 1914-1921.

(6) Shimizu, T.; Hino, A.; Tsutsumi, A.; Park, Y. K.; Watanabe, W.; Kurokawa, M. Anti-influenza virus activity of propolis in vitro and its efficacy against influenza infection in mice. Antivir Chem Chemother. 2008, 19, 7-13.

(7) Silici, S.; Koç, N. A.; Ayangil, D.; Cankaya, S. Antifungal activities of propolis collected by different races of honeybees against yeasts isolated from patients with superficial mycoses. J Pharmacol Sci. 2005, 99, 39-44.

(8) Grunberger, D.; Banerjee, R.; Eisinger, K.; Oltz, E. M.; Efros, L.; Caldwell, M.; Estevez, V.; Nakanishi, K. Preferential cytotoxicity on tumor cells by caffeic acid phenethyl ester isolated from propolis. Experientia 1988, 44, 230-232.

(9) Medana, C.; Carbone, F.; Aigotti, R.; Appendino, G.; Baiocchi, C. Selective analysis of phenolic compounds in propolis by HPLC-MS/MS. Phytochem Anal. 2008, 19, 32-39.

(10) Chen, C. N.; Wu, C. L.; Shy, H. S.; Lin, J. K. Cytotoxic prenylflavanones from Taiwanese propolis. J. Nat. Prod. 2003, 66, 503-506.

(11) Chen, C. N.; Wu, C. L.; Lin, J. K. Propolin C from propolis induces apoptosis through activating caspases, Bid and cytochrome c release in human melanoma cells. Biochem. Pharmacol. 2004, 67, 53-66.

(12) Chen, C. N.; Weng, M. S.; Wu, C. L.; Lin, J. K. Comparison of radical scavenging activity, cytotoxic effects and apoptosis induction in human melanoma cells by Taiwanese propolis from different sources. Evid. Based Complement. Alternat. Med. 2004, 1, 175-185.

(13) Chen, C. N.; Wu, C. L.; Lin, J. K. Apoptosis of human melanoma cells induced by the novel compounds propolin A and propolin B from Taiwanese propolis. Cancer Lett. Cancer Lett. 2007, 245, 218-231.

(14) Huang, W. J.; Huang, C. H.; Wu, C. L.; Lin, J. K.; Chen, Y. W.; Lin, C. L.; Chuang, S. E.; Huang, C. Y.; Chen, C. N. Propolin G, a prenylflavanone, isolated from Taiwanese propolis, induces caspase-dependent apoptosis in brain cancer cells. J. Agric. Food. Chem. 2007, 55, 7366-7376.

(15) Weng, M. S; Liao, C. H.; Chen, C. N.; Wu, C. L.; Lin, J. K. Propolin H from Taiwanese propolis induces G1 arrest in human lung carcinoma cells. J. Agric. Food. Chem. 2007, 55, 5289-5298.

(16) Chen, Y. W.; Wu, S. W.; Ho, K. K.; Lin, S. B.; Huang, C. Y.; Chen, C. N. Characterization of Taiwanese propolis collected from different seasons and locations. J. Sci. Food Agric. 2008, 88, 412-419.

(17) Kumazawa, S.; Goto, H., Hamasaka, T., Fukumoto, S., Fujimoto, T., Nakayama, T. A. A new prenylated flavonoid from propolis collected in Okinawa, Japan. Biosci., Biotechnol. Biochem. 2004, 68, 260-262.

(18) Kumazawa, S.; Ueda, R., Hamasaka, T., Fukumoto, S., Fujimoto, T., Nakayama, T. Antioxidant prenylated flavonoids from propolis collected in Okinawa, Japan. J. Agric. Food Chem. 2007, 55, 7722-7725.

(19) Duvic, M.; Vu, J. Vorinostat: a new oral histone deacetylase inhibitor approved for cutaneous T-cell lymphoma. Expert Opin Investig Drugs. 2007, 16, 1111-1120.

(20) Atmaca, A.; Al-Batran, S. E.; Maurer, A.; Neumann, A.; Heinzel, T.; Hentsch, B.; Schwarz, S. E.; Hövelmann, S.; Göttlicher, M.; Knuth, A.; Jäger, E. Valproic acid (VPA) in patients with refractory advanced cancer: a dose escalating phase I clinical trial. Br J Cancer. 2007, 97, 177-182.

(21) Gojo, I.; Jiemjit, A.; Trepel, J. B.; Sparreboom, A.; Figg, W. D.; Rollins, S.; Tidwell, M. L.; Greer, J.; Chung, E. J.; Lee, M. J.; Gore, S. D.; Sausville, E. A.; Zwiebel, J.; Karp, J. E. Phase 1 and pharmacologic study of MS-275, a histone deacetylase inhibitor, in adults with refractory and relapsed acute leukemias. Blood. 2007, 109, 2781-2790.

(22) Hadnagy, A.; Beaulieu, R.; Balicki, D. Histone tail modifications and noncanonical functions of histones: perspectives in cancer epigenetics. Mol Cancer Ther. 2008, 7, 740-748.

(23) Mariadason, J. M. HDACs and HDAC inhibitors in colon cancer. Epigenetics. 2008, 3, 28-37.

(24) McLaughlin, F.; La Thangue, N. B. Histone deacetylase inhibitors open new doors in cancer therapy. Biochem Pharmacol. 2004, 68, 1139-1144.

(25) Villar-Garea, A.; Esteller, M. Histone deacetylase inhibitors: understanding a new wave of anticancer agents. Int J Cancer 2004, 112, 171-178.

(26) Shankar, S.; Srivastava, R. K. Histone deacetylase inhibitors: mechanisms and clinical significance in cancer: HDAC inhibitor-induced apoptosis. Adv Exp Med Biol. 2008, 615, 261-298.

(27) Pan, L. N.; Lu, J.; Huang, B. HDAC inhibitors: a potential new category of anti-tumor agents. Cell Mol Immunol. 2007, 4, 337-343.

(28) Cao, G.; Sofic, E.; Prior, R. L. Free Radic. Biol. Med. 1997, 22, 749-760.

(29) Chen, Y. W. et al., J. Sci. Food Agric. 2007, DOI: 1002/jsfa.

(30) Wu, X. et al., Int J Neuropsychopharmacol. 2008 Jul. 9: 1-12.

(31) Leng Y. et al., J Neurosci. 2008 Mar. 5; 28(10):2576-88.

(32) Gray S G and Dangond F. Epigenetics. 2006 April-June; 1(2):67-75. Epub 2006 Mar. 5.

(33) Sandri-Vakili G. et al., Hum Mol Genet. 2007 Jun. 1; 16(11):1293-306. Epub 2007.

(34) Hahnen E. et al., J Neurochem. 2006 July; 98(1):193-202.

(35) Riessland M. et al., Hum Genet. 2006 August; 120(1):101-10. Epub 2006 May.

(36) Kernochan L E et al., Hum Mol Genet. 2005 May 1; 14(9):1171-82. Epub 2005 March.

(37) Minamiyama M et al., Hum Mol Genet. 2004 Jun. 1; 13(11):1183-92. Epub 2004 April.

(38) Corcoran L J et al., Curr Biol. 2004 Mar. 23; 14(6):488-92.

(39) Hockly E et al., Proc Natl Acad Sci USA. 2003 Feb. 18; 100(4):2041-6. Epub 2003.

(40) Sadri-Vakili G et al., Hum Mol Genet. 2007 Jun. 1; 16(11):1293-306. Epub 2007 April.

1-9. (canceled)

10. A method for inhibiting growth of cancer cells in a subject in need thereof, comprising administering to the subject a compound or a pharmaceutical composition containing the compound and a pharmaceutically acceptable excipient or carrier, wherein the compound is represented by a formula selected from the group consisting of:

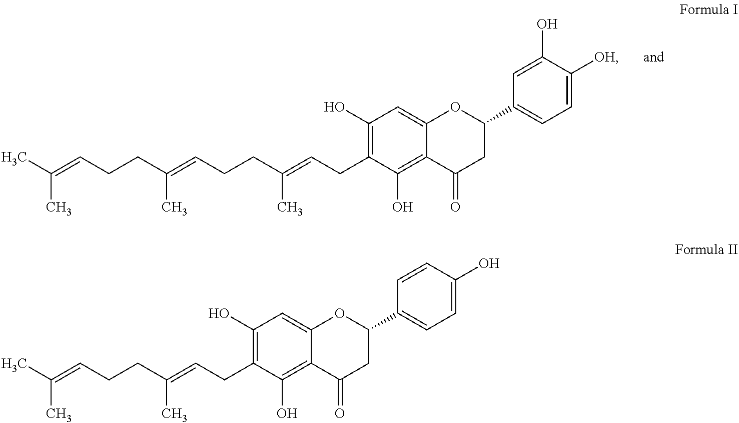

11. The method of claim 10, wherein the cancer cells are breast cancer cells

12. A method for treating a disease in association with histone deacetylation in a subject in need thereof, comprising administering to the subject a compound or a pharmaceutical composition containing the compound and a pharmaceutically acceptable excipient or carrier, wherein the compound is represented by a formula selected from the group consisting of:

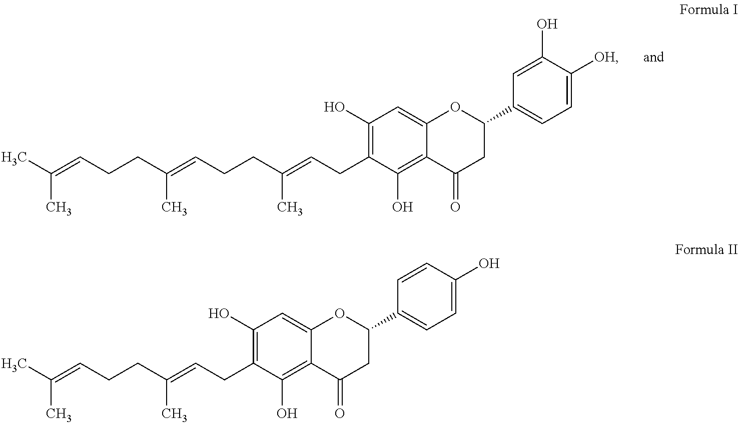

13. A method for providing neuroprotective effects or treating a neurodegenerative disease in a subject in need thereof, comprising administering to the subject a compound or a pharmaceutical composition containing the compound and a pharmaceutically acceptable excipient or carrier, wherein the compound is represented by a formula selected from the group consisting of:

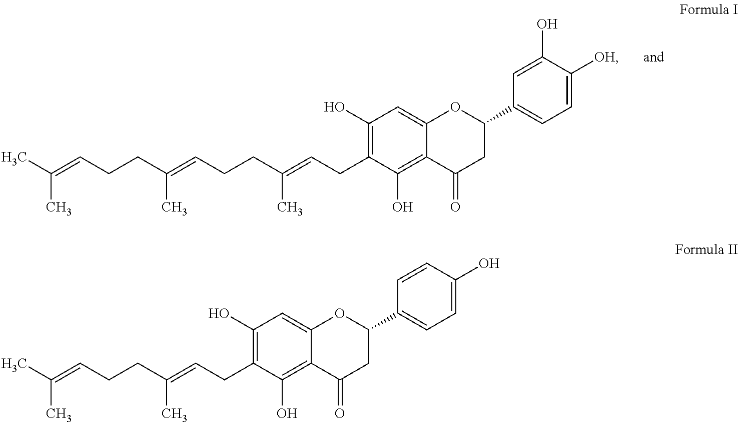

14. The method of claim 13, wherein the subject has a neurodegenerative disease.

15. The method of claim 14, wherein the neurodegenerative disease is selected from the group consisting of multiple sclerosis (MS), Huntington's disease (HD), spinal muscular atrophy (SMA), spinal and bullar muscular atrophy (SBMA), and amyotrophic lateral sclerosis (ALS).
